(19) United States
(12) Patent Application Publication
(10) Pub. No.: US 2016/0355864 A1
(43) Pub. Date: Dec. 8, 2016

(54) METHOD FOR DETERMINING WHETHER OR NOT TEST SAMPLE CONTAINS PHYTOPATHOGENIC OOMYCETE

(71) Applicant: Panasonic Intellectual Property Management Co., Ltd., Osaka (JP)

(72) Inventor: YOSHITSUGU URIU, Nara (JP)

(21) Appl. No.: 14/846,700

(22) Filed: Sep. 4, 2015

(30) Foreign Application Priority Data
Jun. 2, 2015 (JP) 2015-111885

(51) Int. Cl. C12Q 1/04

(52) U.S. Cl. CPC C12Q 1/04

(57) ABSTRACT

Provided is a method for determining whether or not a test sample contains a phytopathogenic oomycete selectively from two kinds of oomycetes of a phytopathogenic oomycete and a non-phytopathogenic oomycete. The present method comprises: (a) putting the test sample on a front surface of a film comprising a through-hole having a cross-sectional area of not less than 0.785 square micrometers and not more than 7.065 square micrometers; (b) leaving the test sample at rest after the step (a); (c) observing a back surface of the film after the step (b); and (d) determining that the test sample contains the phytopathogenic oomycete, if an oomycete is found on the back surface of the film in the step (c).

### BACKGROUND

### 1. Technical Field

The present invention relates to a method for determining whether or not a test sample contains a phytopathogenic oomycete.

### 2. Description of the Related Art

Japanese Patent Application laid-open Publication No. 2005-287337A discloses a method for counting the number of mold cells in a specimen by the culture for a short time and capable of accurately counting the cell number. FIG. 14 shows a cross-sectional view of a microporous membrane supporting material used for the method disclosed therein. According to this method, the extended multiple pseudomycelia of a mold cell 13 cultured by a liquid culture or a mold cell 13 cultured on a microporous membrane 1 of a microporous membrane supporting material 4 are photographed 5 and the shape, area and luminous intensity are recognized and analyzed by an image analytic means 10. The number of the mold cells 13 can be counted by the culture for a short time. The microporous membrane 1 is interposed between a pressing ring 2 and a base 3.

### SUMMARY

An object of the present invention is to provide a method for determining whether or not a test sample contains a phytopathogenic oomycete selectively from two kinds of oomnycetes of a phytopathogenic oomycete and a non-phytopathogenic oomycete.

The present invention is a method for determining whether or not a test sample contains a phytopathogenic oomycete, the method comprising:

(a) putting the test sample on a front surface of a film comprising a through-hole having a cross-sectional area of not less than 0.785 square micrometers and not more than 7.065 square micrometers;

(b) leaving the test sample at rest after the step (a);

(c) observing a back surface of the film after the step (b); and

(d) determining that the test sample contains the phytopathogenic oomycete, if an oomycete is found on the back surface of the film in the step (c).

The present invention provides a method for determining whether or not a test sample contains a phytopathogenic oomycete selectively from two kinds of oomycetes of a phytopathogenic oomycete and a non-phytopathogenic oomycete.

### BRIEF DESCRIPTION OF THE DRAWINGS

FIG. 1 shows a cross-sectional view of a container.

FIG. 2 shows a cross-sectional view of a film.

FIG. 3 shows a cross-sectional view of the container to which a test sample has been supplied.

FIG. 4 shows a cross-sectional view of the film having a front surface on which a phytopathogenic oomycete has been put.

FIG. 5 is a cross-sectional view showing how the phytopathogenic oomycete penetrates the film.

FIG. 6 shows a cross-sectional view of one example of a method for accelerating the incubation of the oomycete.

FIG. 7 shows a cross-sectional view, subsequently to FIG. 6, of one example of a method for accelerating the incubation of the oomycete.

FIG. 8 is a cross-sectional view showing how to observe the oomycete from the back surface of the film.

FIG. 9 is a cross-sectional view showing how to observe the oomycete from the back surface of the film.

FIG. 10 is a microscope photograph of the back surface of the film in the inventive example 1A,

FIG. 11 is a microscope photograph of the back surface of the film in the inventive example 1B.

FIG. 12 is a microscope photograph of the back surface of the film in the comparative example 1A.

FIG. 13 is a microscope photograph of the back surface of the in the comparative example 1B.

FIG. 14 shows a cross-sectional view of the rnicroporous membrane supporting material used for the method for counting the number of mold cells disclosed in Japanese Patent Application laid-open Publication No. 2005-287337A.

### DETAILED DESCRIPTION OF THE EMBODIMENT

First, an oomycete will be described. Oomycetes are roughly divided into a phytopathogenic oomycete and a non-phytopathogenic oomycete. An example of the phytopathogenic oomycete is Pythium helicoides or Pythium aphaniderrnatum. These phytopathogenic oomycetes cause pythium red blight and a root rot disease. First, these phytopathogenic oomycetes infect a root of a plant. Then, these phytopathogenic oomycetes cause the root of the plant to rot. Finally, these phytopathogenic oomycetes kill the plant. An example of the non-phytopathogenic oomycete is Pythium dissotocum, Pythium catenulatum, Pythium torulosum or Pythium inflatum. Pythium dissotocum may be classified as a weak-phytopathogenic oomycete. In the instant specification, the weak-phytopathogenic oomycete is classified as a non-phytopathogenic oomycete. In other words, the word “non-phytopathogenic oomycete” includes a weak-phytopathogenic oomycete. The word “phytopathogenic oomycete” does not include a weak-phytopathogenic oomycete.

The term “phytopathogenic” means to have pathogenicity to plants. The term “non-phytopathogenic” means not to have pathogenicity to plants. Even if an oomycete has pathogenicity, however, if the oomycete has no pathogenicity to plants, the oomycete is non-phytopathogenic. In other words, if an oomycete does not have adverse effects on plants, the oomycete is non-phytopathogenic. The prefix “non-” included in the term “non-phytopathogenic” does not modify “phyto”. The prefix non-modifies “pathogenic”.

Hereinafter, the embodiment of the present invention will be described in more detail with reference to the drawings.

(Step (a))

In the step (a), a test sample is put on a front surface of a flan comprising a through-hole having a cross-sectional area of not less than 0.785 square micrometers and not more than 7.065 square micrometers.

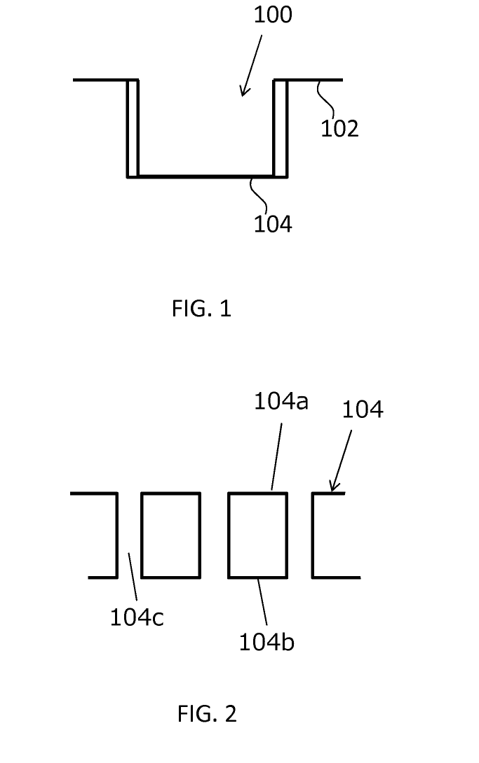

In particular, as shown in FIG. 1, a container 100 is prepared. It is desirable that the container 100 comprises a flange 102 at the upper end thereof. The bottom of the container 100 is formed of a film 104. An example of the material of the film 104 is organic resin such as polyethylene terephthalate.

FIG. 2 shows a cross-sectional view of the film 104. The film 104 has a front surface 104a, a back surface 104b, and a through-hole 104c. One of the characteristics of the present invention is a cross-sectional area of the through-hole 104c.

The through-hole 104c has a cross-sectional area of not less than 0.785 square micrometers and not more than 7.065 square micrometers. In particular, it is desirable that the through-hole 104c has a shape of a cylinder having a diameter of not less than micrometer and not more than 3 micrometers. The importance of these cross-sectional area and diameter will be described later.

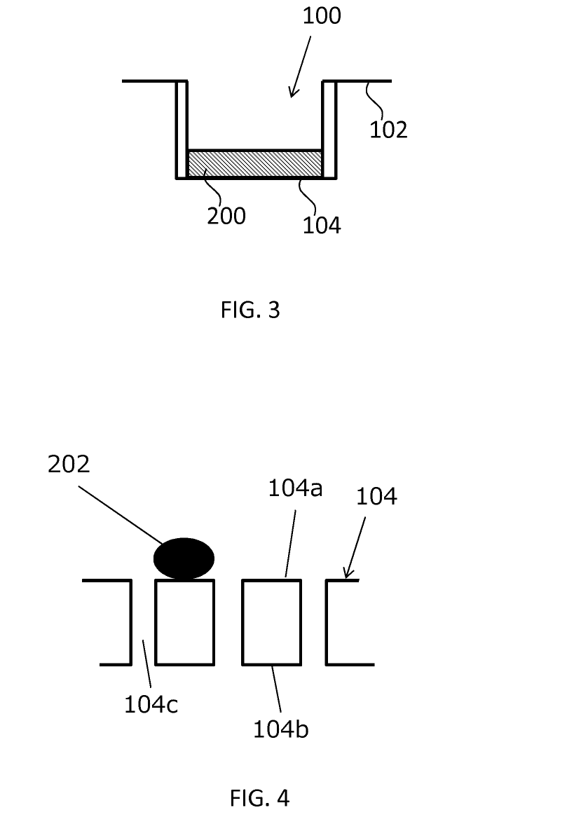

As shown in FIG. 3, a test sample 200 is supplied to the inside of this container 100. In this way, the test sample 200 is put on the front surface 104a of the film 104. When the test sample 200 contains a phytopathogenic oomycete 202, the phytopathogenic oomycete 202 is put on the front surface 104a of the film 104, as shown in FIG. 4.

The test sample 200 is solid, liquid, or gaseous. It is desirable that the test sample 200 is solid or liquid. An example of the solid test sample 200 is soil or a crushed plant. Another example is an agricultural material such as vermiculite, rock wool or urethane, An example of the liquid test sample 200 is agricultural water, a solution used for hydroponic culture, a liquid used to wash a plant, a liquid extracted from a plant, a liquid used to wash an agricultural material, or a liquid used to wash clothing or shoes of a worker.

(Step (b))

In the step (b), the test sample 200 is left at rest for a certain time after the step (a). The importance of the cross-sectional area or the diameter of the through-hole 104c will be described below.

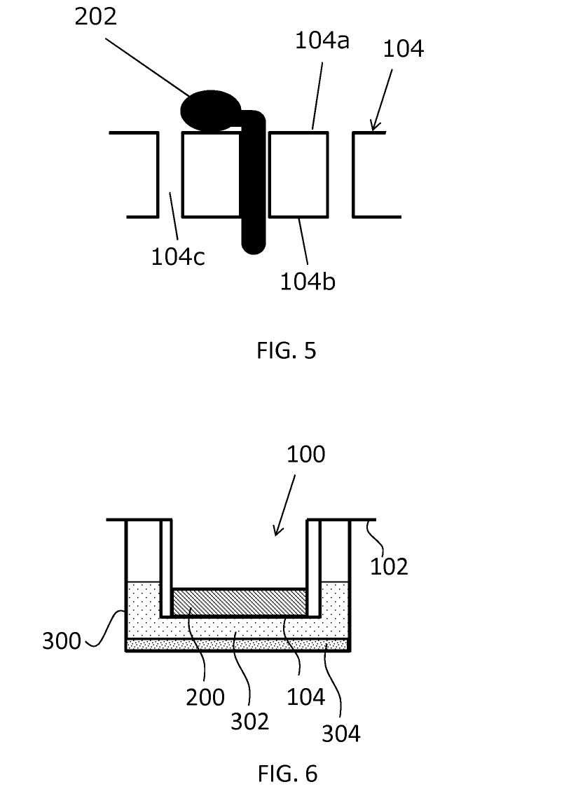

In the step (b), various oomycetes contained in the test sample 200 are grown. When the through-hole 104c has a cross-sectional area of not less than 0.785 square micrometers and not more than 7.065 square micrometers, as shown in FIG. 5, the phytopathogenic oomycete 202 grows up so as to penetrate the through-hole 104c. This is demonstrated in the inventive examples and the comparative examples which will be described later. As a result, the phytopathogenic oomycete 202 appears on the back surface 104b of the film 104. On the other hand, within this range of the cross-sectional area, the non-phytopathogenic oomycete does not penetrate the through-hole 104c. For this reason, the non-phytopathogenic oomycete does not appear on the back surface 104b of the film 104. In this way, the phytopathogenic oomycete 202 appears on the back surface 104b selectively. In other words, the phytopathogenic oomycete 202 appears outside of the container 100 selectively.

When the through-hole 104c has a cross-sectional area less than 0.785 square micrometers, neither the non-phytopathogenic oomycete nor the phytopathogenic oomycete penetrates the through-hole 104c. On the other hand, when the through-hole 104c has a cross-sectional area more than 7.065 square micrometers, not only the phytopathogenic oomycete but also the non-phytopathogenic oomycete penetrates the through-hole 104c. For this reason, the selectivity is lost when the through-hole 104c has a cross-sectional area less than 0.785 square micrometers or more than 7.065 square micrometers.

The thickness of the film 104 is not limted, as far as the phytopathogenic oomycete 202 appears outside of the container 100 selectively. The film 104 may have a thickness of not less than 10 micrometers and not more than 100 micrometers. It is desirable that the film 104 has plural through-holes 104c, as shown in FIG. 3-FIG. 5.

A culture medium may be supplied to the test sample 200 to accelerate the incubation of the oomycete. In particular, a culture medium may be supplied to the inside of the container 100 containing the test sample 200. It is desirable that the culture medium is liquid. The culture medium may be supplied in the step (b). Alternatively, the culture medium may be supplied prior to the step (b). In other words, the culture medium may be supplied in the step (a). The culture medium may be supplied to the inside of the container 100 prior to the step (a).

FIG. 6 shows another method for accelerating the incubation of the oomycete. As shown in FIG. 6, it is desirable that the back surface 104b of the film 104 is in contact with a liquid culture medium 302. First, a second container 300 having the liquid culture medium 302 therein is prepared. Hereinafter, the container 100 is referred to as “first container 100” to distinguish it from the second container 300. The first container 100 is stacked on the second container 300 in such a manner that the lower surface of the flange 102 is in contact with the upper end of the second container 300. In other words, the first container 100 is supported by the upper end of the second container 300. In this way, the liquid culture medium 302 is sandwiched between the back surface 104b of the film 104 and the bottom surface of the second container 300.

Alternatively, after the first container 100 is stacked on the second container 300, the liquid culture medium 302 may be supplied between the back surface 104b of the film 104 and the bottom surface of the second container 300.

Since the liquid culture medium 302 is in contact with the back surface 104b of the film 104, the liquid culture medium 302 is soaked up by a capillary phenomenon through the through-hole 104c. In place of the liquid culture medium 302, a viscous solid culture medium may also be used. In this case, when the first container 100 is stacked on the second container 300, the viscous solid culture medium is transformed so as to penetrate the through-hole 104c. In this way, the culture medium 302 reaches the inside of the container 100. By the culture medium 302 which has reached the inside of the container 100, the incubation of the oomycete is accelerated. As shown in FIG. 6, both of a solid culture medium 304 and the liquid culture medium 302 may be used. In this case, the liquid culture medium 302 is sandwiched between the solid culture medium 304 and the film 104.

(Step (c))

In the step (c), the back surface 104b of the film 104 is observed after the step (b). It is desirable that the back surface 104b is observed using an optical microscope.

The phytopathogenic oomycete 202 appears on the back surface 104b of the film 104, as described in the step (b). On the other hand, the non-phytopathogenic oomycete does not appear on the back surface 104b of the film 104. In this way, in the present invention, the phytopathogenic oomycete 202 appears on the back surface 104b of the film 104 selectively.

In other words, the phytopathogenic oomycete 202 penetrates the through-hole 104c, whereas the non-phytopathogenic oomycete does not penetrate the through-hole 104c. For this reason, the non-phytopathogenic oomycete does not appear on the back surface 104b of the film 104. In this way, the phytopathogenic oomycete 202 appears on the back surface 104b selectively. In other words, the phytopathogenic oomycete 202 appears outside of the container 100 selectively.

In the step (c), it is observed whether or not the phytopathogenic oomycete 202 appears on the back surface 104b of the film 104.

In particular. whether or not the phytopathogenic oomycete 202 appears on the back surface 104b of the film 104 is observed as below.

First, the test sample is turned into a gel. In more detail, an agarose aqueo solution is supplied to the first container 100. Then, the agarose aqueous solution containing the test sample is stirred. Finally, the test sample is left at rest at room temperature. In this way, the test sample is turned into a gel.

Then, the first container 100 is drawn up from the second container 300. Prior to the gelation, the first container 100 may be drawn up from the second container 300.

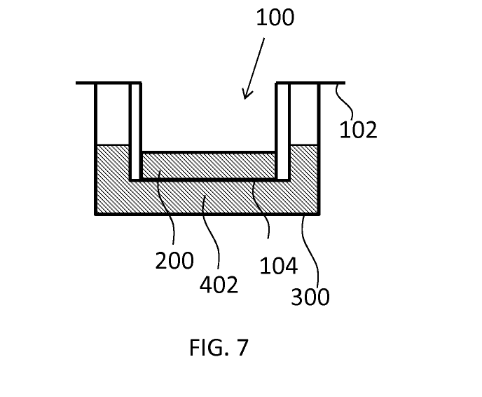

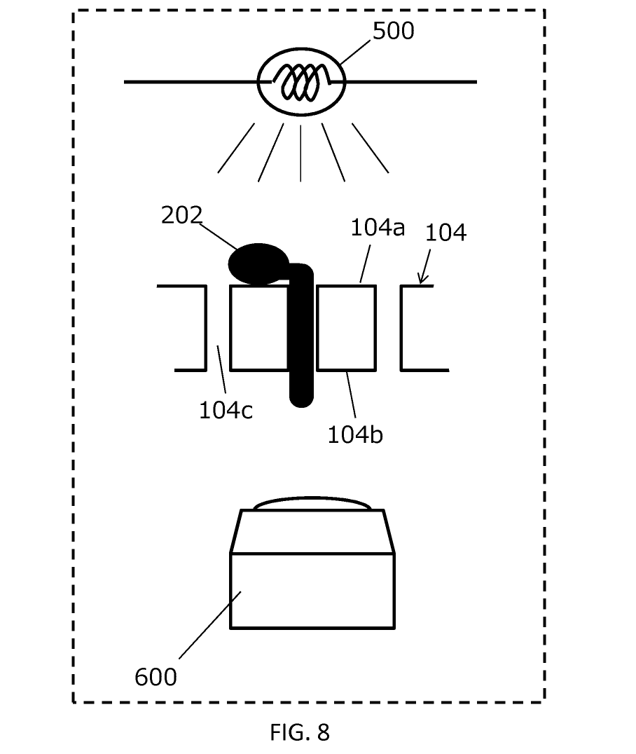

The liquid culture medium 302 and the solid culture medium 304 are removed from the second container 300. Then, a fluorescent agent having oomycete combining ability is added to the inside of the second container 300. Hereinafter, such a fluorescent agent is referred to as “oomycete fluorescent agent”. The reference number of the oomycete fluorescent agent is 402. Then, as shown in FIG. 7, the first container 100 is stacked on the second container 300 having the oomycete fluorescent agent 402 therein. Alternatively, The oomycete fluorescent agent 402 may be supplied between the back surface 104b of the film 104 and the bottom surface of the second container 300 after the first container 100 is stacked on the second container 300.

A part of the phytopathogenic oomycete 202 which has appeared on the back surface 104b of the film 104 is dyed with the oomycete fluorescent agent 402. Since the test sample 200 has been turned into a gel, the oomycete fluorescent agent 402 does not spread into the first container 100. For this reason, the non-phytopathogenic oomycete contained in the first container 100 is not dyed with the oomycete fluorescent agent 402.

As shown in FIG. 8, the thus-dyed phytopathogenic oomycete 202 is observed using a microscope 600 located under the back surface 104b of the film 104, while the film 104 is irradiated with light using a light source 500 located over the front surface 104a of the film 104.

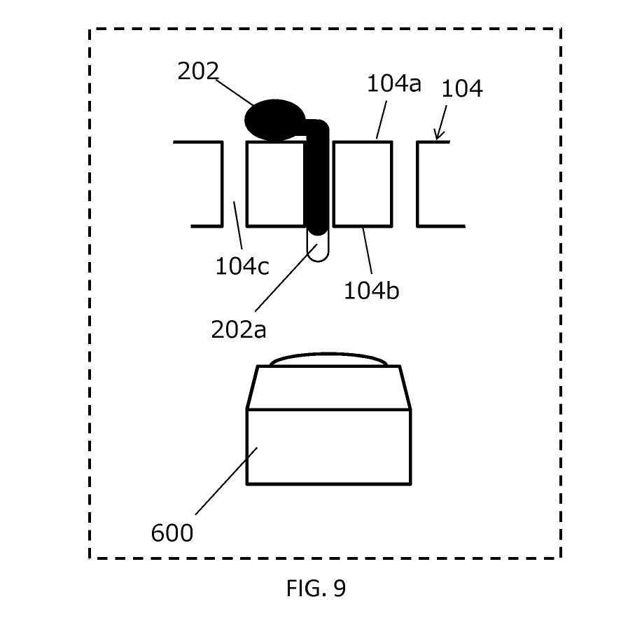

In place of the oomycete fluorescent agent 402, a fluorescent agent having oomycete combining ability may also be used. In this case, a part 202a of the phytopathogenic oomycete 202 which has appeared on the back surface 104b of the film 104 is dyed with the fluorescent agent having oomycete combining ability. As shown in FIG. 9, the phytopathogenic oomycete 202 dyed with the fluorescent agent having oomycete combining ability is observed using the microscope 600 located under the back surface 104b of the film 104.

(Step (d))

In the step (d), it is determined that the test sample contains a phytopathogenic oomycete, if an oomycete is found on the back surface 104b of the film 104 in the step (c). Needless to say, it is determined that the test sample does not contain a phytopathogenic oomycete, if an oomycete is not found on the back surface 104b of the film 104 in the step (c).

### EXAMPLES

The present invention will be described in more detail with reference to the following examples.

(Incubation of Pythium helicoides)

Pythium helicoides, one of phytopathogenic oomycetes, was inoculated on a cornmeal agar culture medium together with dried turfgrass. Then, the culture medium was left at rest at a temperature of 25 degrees Celsius for 24 hours. Pythium helicoides was given by Professor Kageyarna, who belongs to Gifu University River Basin Research Center. The dried turfgrass was provided by drying Korean lawn grass sterilized in accordance with a high temperature and high pressure sterilization method at 60 degrees Celsius for approximately 24 hours.

Then, the dried turfgrass to which a pseudomycelium was adhered was picked up from the culture medium. The thus-picked dried turfgrass was provided afloat to the pure water contained in a petri dish. The volume of the pure water was 20 milliliters.

After 18 hours, the water contained in the petri dish was observed using an optical microscope. As a result, the present inventors confirmed that zoospores of Pythium helicoides were released in the water contained in the petri dish. In this way, an aqueous solution containing Pythium helicoides was provided. Hereinafter, this aqueous solution is referred to as “phytopathogenic aqueous solution”.

(Preparation of Culture Medium)

A potato dextrose agar culture medium melted at a high temperature was added to the second container 300. The potato dextrose agar culture medium had a volume of 250 microliters. Then, the potato dextrose agar culture medium was turned into a gel at room temperature. In this way, the potato dextrose agar culture medium gel was provided as the solid culture medium 304.

A hydroponic culture solution (e.g., Otsuka-SA nutrient solution) having a volume of 350 microliters was added as the liquid culture medium 302 to the second container 300 containing the potato dextrose agar culture medium gel. In this way, the second container 300 containing the liquid culture medium 302 and the solid culture medium 304 was prepared.

### Inventive Example 1A

The first container 100 shown in FIG. 1 was prepared. This first container 100 was made of plastic. As shown in FIG. 2, the bottom surface of the first container 100 was formed of a polyethylene terephthalate film 104 (available from Merck KGaA, trade name: Millicell PISP 12R 48). This polyethylene terephthalate film 104 comprised plural through-holes 104c each having a diameter of 3 micrometers. The plural through-holes 104c were provided randomly in the film 104.

Then, as shown in FIG. 6, the first container 100 was stacked on the second container 300. The back surface 104b of the film 104 was in contact with the liquid culture medium 302. Subsequently, the hydroponic culture solution having a volume of 200 microliters was added to the inside of the first container 100. Furthermore, the phytopathogenic aqueous solution containing 200 zoospores of Pythium helicoides was added to the inside of the first container 100.

The first container 100 was left at rest at a temperature of 25 degrees Celsius for 6 hours.

Subsequently, the first container 100 was separated from the second container 300. The phytopathogenic aqueous solution contained in the first container 100 was removed. Then, an agarose aqueous solution having a concentration of 2% was added to the inside of the first container 100. The agarose aqueous solution was turned into a gel at room temperature.

A fluorescent agent having oomycete combining ability (available from Beckton Dickinson and Company, trade name: Calcofluor White (BD261195)) having a volume of 600 milliliters was added to the inside of the second container 300. The final concentration of the fluorescent agent having oomycete combining ability was 0.005%.

Then, the first container 100 was stacked on the second container 300 again. The back surface 104b of the film 104 was in contact with the fluorescent agent having oomycete combining ability. The first container 100 was left at rest at 25 degrees Celsius for 10 minutes. Since the gel was located in the first container 100, the fluorescent agent having oomycete combining ability did not spread into the first container 100.

Subsequently, the first container 100 was separated from the second container 300. The fluorescent agent having oomycete combining ability contained in the second container 300 was removed. Then, a buffer solution was added to the inside of the second container 300. The following Table 1 shows components contained in this buffer solution and their concentrations.

TABLE 1

| Component | Concentration (mmol/L) |
|---|---|
| NaCl | 137 |
| KCl | 2.7 |
| Na₂HPO₄ | 10 |
| KH₂PO₄ | 1.76 |

As shown in FIG. 9, the back surface 104b of the film 104 was observed using a fluorescent microscope 600 (available from Molecular Devices Japan K.K. Trade name: ImageXpress MICRO). Table 2 shows filters and a lens used for the fluorescent microscope 600.

TABLE 2

| | |
|---|---|
| Excitation | Band pass filter having a center wavelength of 377 |
| filter | nanometers and a band width of 11 nanometers |
| Fluorescence | Band pass filter having a center wavelength of 447 |
| filter | nanometers and a band width of 60 nanometers |
| Object lens | Magnification: 10 times/Numerical aperture: 0.30 |

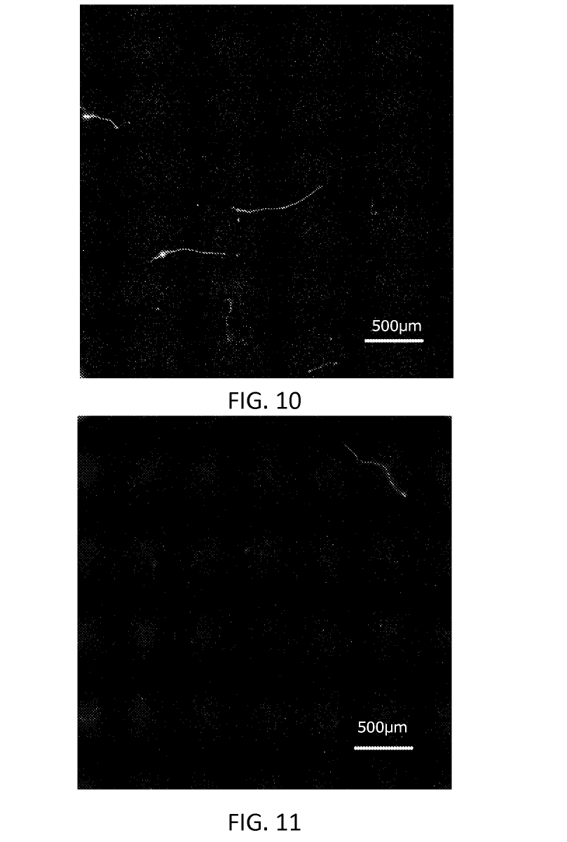

FIG. 10 is a microscope photograph of the back surface 104b of the film 104 in the inventive example 1A. As seen in FIG. 10, pseudohyphae of Pythium helicoides appear on the back surface 104b. This means that the pseudohyphae of Pythium helicoides penetrated the through-hole 104c.

The number of the pseudohyphae of Pythium helicoides which appeared on the back surface 104b was counted visually. The inventive example 1A was repeated two times-three times. As a result, the mean value of the number of the pseudohyphae of Pythium helicoides which appeared on the back surface 104b was 18.0.

### Inventive Example 1B

An experiment similar to the inventive example 1A was conducted, except that each of the through-holes 104c had a diameter of 1 micrometer. In particular, a polyethylene terephthalate film 104 (available from Merck KGaA, trade name: Millicell PIRP 12R 48) was used.

FIG. 11 is a microscope photograph of the back surface 104b of the film 104 in the inventive example 1B. As seen in FIG. 11, pseudohyphae of Pythium helicoides appear on the back surface 104b. This means that the pseudohyphae of Pythium helicoides penetrated the through-hole 104c.

### Comparative Example 1A

An experiment similar to the inventive example 1A was conducted, except that each of the through-holes 104c had a diameter of 0.4 micrometers. In particular, a polyethylene terephthalate film 104 (available from Merck KGaA, trade name: Millicell PIHT 12R 48) was used.

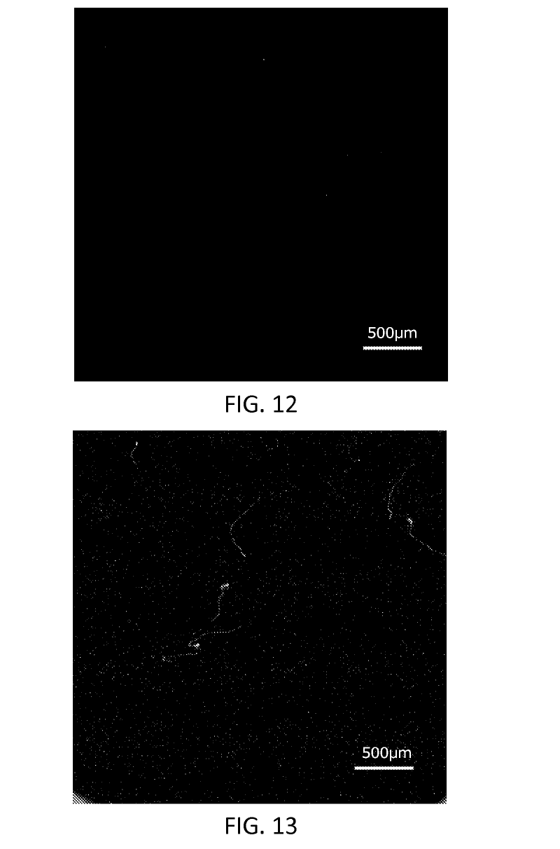

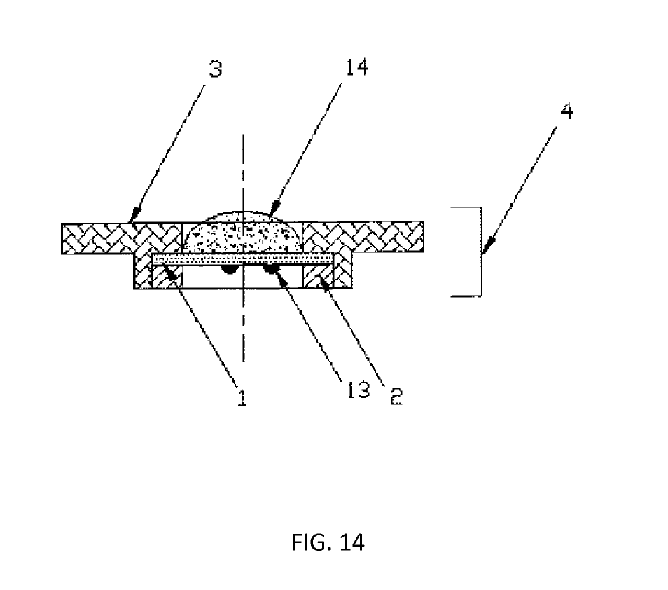

FIG. 12 is a microscope photograph of the back surface 104b of the film 104 in the comparative example 1A. As seen in FIG. 12, pseudohyphae of Pythium helicoides did not appear on the back surface 104b. This means that the pseudohyphae of Pythium helicoides did not penetrate the through-hole 104c.

### Comparative Example 1B

An experiment similar to the inventive example 1A was conducted, except that each of the through-holes 104c had a diameter of 8 micrometers. In particular, a polyethylene terephthalate film 104 (available from Merck KGaA, trade name: Millicell PIEP 12R 48) was used.

FIG. 13 is a microscope photograph of the back surface 104b of the film 104 in the comparative example 1B. As seen in FIG. 13, pseudohyphae of Pythium helicoides appear on the back surface 104b. This means that the pseudohyphae of Pythium helicoides penetrated the through-hole 104c.

### Inventive Example 2A

In the inventive examples 2A-2B and the comparative examples 2A-2B, a phytopathogenic aqueous solution containing zoospores of Pythium myliotaerum was used in place of the phytopathogenic aqueous solution containing zoospores of Pythium helicoides. Similarly to Pythium helicoides, Pythium myliotaerum is also one kind of phytopathogenic oomycete. A phytopathogenic aqueous solution containing zoospores of Pythium myliotaerum was prepared similarly to the case of the phytopathogenic aqueous solution containing zoospores of Pythium helicoides.

In the inventive example 2A, an experiment similar to the inventive example 1A was conducted, except that the aqueous solution contained not Pythium helicoides but Pythium myliotaerum. The through-hole 104c had a diameter of 3 micrometers.

### Inventive Example 2B

In the inventive example 2B, an experiment similar to the inventive example 1B was conducted, except that the aqueous solution contained not Pythium helicoides but Pythium myliotaerum. The through-hole 104c had a diameter of 1 micrometer.

### Comparative Example 2A

In the comparative example 2A, an experiment similar to the comparative example 1A was conducted, except that the aqueous solution contained not Pythium helicoides but Pythium myliotaerum. The through-hole 104c had a diameter of 0.4 micrometers.

### Comparative Example 2B

In the comparative example 2B, an experiment similar to the comparative example 1B was conducted, except that the aqueous solution contained not Pythium helicoides but Pythium myliotaerum. The through-hole 104c had a diameter of 8 micrometers.

### Inventive Example 3A

In the inventive examples 3A-3B and the comparative examples 3A-3B, a phytopathogenic aqueous solution containing zoospores of Pythium aphanidermatum was used in place of the phytopathogenic aqueous solution containing zoospores of Pythium helicoides. Similarly to Pythium helicoides, Pythium aphanidermatum is also one kind of phytopathogenic oomycete. A phytopathogenic aqueous solution containing zoospores of Pythium aphanidermatum was prepared similarly to the case of the phytopathogenic aqueous solution containing zoospores of Pythium helicoides.

In the inventive example 3A, an experiment similar to the inventive example 1A was conducted, except that the aqueous solution contained not Pythium helicoides but Pythium aphanidermatum. The through-hole 104c had a diameter of 3 micrometers.

### Inventive Example 3B

In the inventive example 3B, an experiment similar to the inventive example 1B was conducted, except that the aqueous solution contained not Pythium helicoides but Pythium aphanidermatum. The through-hole 104c had a diameter of 1 micrometer.

### Comparative Example 3A

In the comparative example 3A, an experiment similar to the comparative example 1A was conducted, except that the aqueous solution contained not Pythium helicoides but Pythium aphanidermatum. The through-hole 104c had a diameter of 0.4 micrometers.

### Comparative Example 3B

In the comparative example 3B, an experiment similar to the comparative example 1B was conducted, except that the aqueous solution contained not Pythium helicoides but Pythium aphanidermatum. The through-hole 104c had a diameter of 8 micrometers.

### Inventive Example 4A

In the inventive examples 4A-4B and the comparative examples 4A-4B, a phytopathogenic aqueous solution containing zoospores of Phytophthora nicotianae was used in place of the phytopathogenic aqueous solution containing zoospores of Pythium helicoides. Similarly to Pythium helicoides, Phytophthora nicotianae is also one kind of phytopathogenic oomycete. A phytopathogenic aqueous solution containing zoospores of Phytophthora nicotianae was prepared similarly to the case of the phytopathogenic aqueous solution containing zoospores of Pythium helicoides.

In the inventive example 4A, an experiment similar to the inventive example 1A was conducted, except that the aqueous solution contained not Pythium helicoides but Phytophthora nicotianae. The through-hole 104c had a diameter of 3 micrometers.

### Inventive Example 4B

In the inventive example 4B, an experiment similar to the inventive example 1B was conducted, except that the aqueous solution contained not Pythium helicoides but Phytophthora nicotianae. The through-hole 104c had a diameter of 1 micrometer.

### Comparative Example 4A

In the comparative example 4A, an experiment similar to the comparative example 1A was conducted, except that the aqueous solution contained not Pythium helicoides but Phytophthora nicotianae. The through-hole 104c had a diameter of 0.4 micrometers.

### Comparative Example 4B

In the comparative example 4B, an experiment similar to the comparative example 1B was conducted, except that the aqueous solution contained not Pythium helicoides but Phytophthora nicotianae. The through-hole 104c had a diameter of 8 micrometers.

### Comparative Example 5A

In the comparative examples 5A-5D, a non-phytopathogenic aqueous solution containing zoospores of Pythium torulosum was used in place of the phytopathogenic aqueous solution containing zoospores of Pythium helicoides. Unlike Pythium helicoides, Pythium torulosum is one kind of non-phytopathogenic oomycete. A non-phytopathogenic aqueous solution containing zoospores of Pythium torulosum was prepared similarly to the case of the phytopathogenic aqueous solution containing zoospores of Pythium helicoides.

In the comparative example 5A, an experiment similar to the inventive example 1A was conducted, except that the aqueous solution contained not Pythium helicoides but Pythium torulosum. The through-hole 104c had a diameter of 3 micrometers.

### Comparative Example 5B

In the comparative example 5B, an experiment similar to the inventive example 1B was conducted, except that the aqueous solution contained not Pythium helicoides but Pythium torulosum. The through-hole 104c had a diameter of 1 micrometer.

### Comparative Example 5C

In the comparative example 5C, an experiment similar to the comparative example 1A was conducted, except that the aqueous solution contained not Pythium helicoides but Pythium torulosum. The through-hole 104c had a diameter of 0.4 micrometers.

### Comparative Example 5D

In the comparative example 5D, an experiment similar to the comparative example 1B was conducted, except that the aqueous solution contained not Pythium helicoides but Pythium torulosum. The through-hole 104c had a diameter of 8 micrometers.

### Comparative Example 6A

In the comparative examples 6A-6D, a non-phytopathogenic aqueous solution containing zoospores of Pythium catenulatum was used in place of the phytopathogenic aqueous solution containing zoospores of Pythium helicoides. Unlike Pythium helicoides, Pythium catenulatum is one kind of non-phytopathogenic oomycete. A non-phytopathogenic aqueous solution containing zoospores of Pythium catenulatum was prepared similarly to the case of the phytopathogenic aqueous solution containing zoospores of Pythium helicoides.

In the comparative example 6A, an experiment similar to the inventive example 1A was conducted, except that the aqueous solution contained not Pythium helicoides but Pythiurn catenulatum. The through-hole 104c had a diameter of 3 micrometers.

### Comparative Example 6B

In the comparative example 6B, an experiment similar to the inventive example 1B was conducted, except that the aqueous solution contained not Pythium helicoides but Pythium catenulatum. The through-hole 104c had a diameter of 1 micrometer.

### Comparative Example 6C

In the comparative example 6C, an experiment similar to the comparative example 1A was conducted, except that the aqueous solution contained not Pythium helicoides but Pythium catenulatum. The through-hole 104c had a diameter of 0.4 micrometers.

### Comparative Example 6D

In the comparative example 6D, an experiment similar to the comparative example 1B was conducted, except that the aqueous solution contained not Pythium helicoides but Pythium catenulatum. The through-hole 104c had a diameter of 8 micrometers.

### Comparative Example 7A

In the comparative examples 7A-7D, a non-phytopathogenic aqueous solution containing zoospores of Pythium inflatum was used in place of the phytopathogenic aqueous solution containing zoospores of Pythium helicoides. Unlike Pythium helicoides, Pythium inflatum is one kind of non-phytopathogenic oomycete. A non-phytopathogenic aqueous solution containing zoospores of Pythium inflatum was prepared similarly to the case of the phytopathogenic aqueous solution containing zoospores of Pythium helicoides.

In the comparative example 7A, an experiment similar to the inventive example 1A was conducted, except that the aqueous solution contained not Pythium helicoides but Pythium inflatum. The through-hole 104c had a diameter of 3 micrometers.

### Comparative Example 7B

In the comparative example 7B, an experiment similar to the inventive example 1B was conducted, except that the aqueous solution contained not Pythium helicoides but Pythium inflatum. The through-hole 104c had a diameter of 1 micrometer.

### Comparative Example 7C

In the comparative example 7C, an experiment similar to the comparative example 1A was conducted, except that the aqueous solution contained not Pythium helicoides but Pythium inflatum. The through-hole 104c had a diameter of 0.4 micrometers.

### Comparative Example 7D

In the comparative example 7D, an experiment similar to the comparative example 1B was conducted, except that the aqueous solution contained not Pythium helicoides but Pythium inflatum. The through-hole 104c had a diameter of 8 micrometers.

The following Table 3-Table 6 show the number of the pseudohyphae which penetrated the through-hole 104c in the inventive example 1A-the comparative example 7D.

TABLE 3

| | (Diameter: 3 micrometers) | |
|---|---|---|
| | | Number of the |
| | | pseudohyphae |
| | | which penetrated |
| | | the through-hole |
| | Name of oomycete | 104c |
| Inventive example 1A | Pythium helicoides | 18.0 |
| Inventive example 2A | Pythium myliotaerum | 48.7 |
| Inventive example 3A | Pythium aphanidermatum | 21.6 |
| Inventive example 4A | Phytophthora nicotianae | 31.8 |
| Comparative example 5A | Pythium torulosum | 0.0 |
| Comparative example 6A | Pythium catenulatum | 0.0 |
| Comparative example 7A | Pythium inflatum | 0.0 |

TABLE 4

| | (Diameter: 1 micrometer) | |
|---|---|---|
| | | Number of the |
| | | pseudohyphae |
| | | which penetrated |
| | | the through-hole |
| | Name of oomycete | 104c |
| Inventive example 1B | Pythium helicoides | 11.0 |
| Inventive example 2B | Pythium myliotaerum | 1.5 |
| Inventive example 3B | Pythium aphanidermatum | 11.6 |
| Inventive example 4B | Phytophthora nicotianae | 3.8 |
| Comparative example 5B | Pythium torulosum | 0.0 |
| Comparative example 6B | Pythium catenulatum | 0.0 |
| Comparative example 7B | Pythium inflatum | 0.0 |

TABLE 5

| | (Diameter: 0.4 micrometers) | |
|---|---|---|
| | | Number of the |
| | | pseudohyphae |
| | | which penetrated |
| | | the through-hole |
| | Name of oomycete | 104c |
| Comparative example 1A | Pythium helicoides | 0.0 |
| Comparative example 2A | Pythium myliotaerum | 0.0 |
| Comparative example 3A | Pythium aphanidermatum | 0.0 |
| Comparative example 4A | Phytophthora nicotianae | 0.0 |
| Comparative example 5C | Pythium torulosum | 0.0 |
| Comparative example 6C | Pythium catenulatum | 0.0 |
| Comparative example 7C | Pythium inflatum | 0.0 |

TABLE 6

| | (Diameter: 8.0 micrometers) | |
|---|---|---|
| | | Number of the |
| | | pseudohyphae |
| | | which penetrated |
| | | the through-hole |
| | Name of oomycete | 104c |
| Comparative example 1B | Pythium helicoides | 59.7 |
| Comparative example 2B | Pythium myliotaerum | 31.3 |
| Comparative example 3B | Pythium aphanidermatum | 17.6 |
| Comparative example 4B | Phytophthora nicotianae | 11.0 |
| Comparative example 5D | Pythium torulosum | 6.0 |
| Comparative example 6D | Pythium catenulatum | 5.5 |
| Comparative example 7D | Pythium inflatum | 23.5 |

As is clear from Table 3 and Table 4, when the through-hole 104c has a diameter of not less than 1 micrometer and not more than 3 micrometers, the phytopathogenic oomycete appears on the back surface 104b of the film 104. On the other hand, within this range of diameter, non-phytopathogenic oomycetes do not appear on the back surface 104b of the film 104. Therefore, the phytopathogenic oomycete 202 appears on the back surface 104b selectively in a case where the through-hole 104c has a diameter of not less than 1 micrometer and not more than 3 micrometers. In other words, the phytopathogenic oomycete 202 appears outside of the container 100 selectively.

As is dear from Table 5, when the through-hole 104c has a diameter of 0.4 micrometers, neither the non-phytopathogenic oomycete nor the phytopathogenic oomycete appears on the back surface 104b of the film 104.

As is dear from Table 6, when the through-hole 104c has a diameter of 8 micrometers, not only the phytopathogenic oomycete but also the non-phytopathogenic oomycete appears on the back surface 104b of the film 104.

Therefore, it is required that the through-hole 104c has a diameter of not less than 1 micrometer and not more than 3 micrometers for the phenomenon that the phytopathogenic oomycete appears on the back surface 104b selectively.

### INDUSTRIAL APPLICABILITY

The present invention can be used to determine easily whether or not a test sample such as agricultural water or soil contains a phytopathogenic oomycete.

### REFERENTIAL SIGNS LIST

100 First container

102 Flange

104 Film

104a Front surface

104b Back surface

104c Through-hole

200 Test sample

202 Phytopathogenic oomycete

202a Part of Phytopathogenic oomycete

300 Second container

302 Liquid culture medium

304 Solid culture medium

402 fluorescent agent having oomycete combining ability

500 Light source

600 Microscope

1. A method for determining whether or not a test sample contains a phytopathogenic oomycete, the method comprising:
(a) putting the test sample on a front surface of a film comprising a through-hole having a cross-sectional area of not less than 0.785 square micrometers and not more than 7.065 square micrometers;
(b) leaving the test sample at rest after the step (a);
(c) observing a back surface of the film after the step (b); and
(d) determining that the test sample contains the phytopathogenic oomycete, if an oomycete is found on the back surface of the film in the step (c).

2. The method according to claim 1, wherein
the phytopathogenic oomycete is phytopathogenic pythium.

3. The method according to claim 1, wherein
the phytopathogenic oomycete is at least one selected from the group consisting of Pythium helicoides, Pythium myliotaerum, Pythium aphanidermatum, and Phytophthora nicotianae.

4. The method according to claim 1, further comprising:
a step of bringing the back surface of the film into contact with a fluorescent agent having oomycete combining ability between the step (b) and the step (c).

5. The method according to claim 4, further comprising:
a step of turning the test sample into a gel before the back surface of the film is brought into contact with the fluorescent agent having oomycete combining ability.

6. The method according to claim 1, further comprising:
a step of supplying a culture medium to the test sample prior to the step (b).

7. The method according to claim 6, wherein
the culture media is a liquid culture media.

8. The method according to claim 6, wherein
the test sample is left at rest while the back surface of the film is in contact with the culture medium in the step (b).

9. The method according to claim 6, wherein
the culture media is a solid culture media.

10. The method according to claim 1, wherein
the film has a thickness of not less than 10 micrometers and not more than 100 micrometers.

11. The method according to claim 1, wherein
the film comprises a plurality of the through-holes.

12. The method according to claim 1, wherein
the test sample is solid.

13. The method according to claim 12, wherein
the solid test sample is at least one selected from the group consisting of soil and a crushed plant.

14. The method according to claim 1, wherein
the test sample is liquid.

15. The method according to claim 14, wherein
the liquid test sample is at least one selected from the group consisting of agricultural water, a liquid used for hydroponic culture, a liquid used for washing a plant, a liquid extracted from a plant, a liquid used for washing an agricultural material, and a liquid used for washing clothing or a shoe.

16. The method according to claim 1, wherein
the phytopathogenic oomycete is phytophthora.
